SEQUENCE LISTING (2 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 14 aa, Homo sapiens
MPSEEGYQDYEPEA

(19) United States
(12) Patent Application Publication
(10) Pub. No.: US 2018/0134776 A1
(43) Pub. Date: May 17, 2018

(54) Alpha-Synuclein Antibodies (4H6)

(71) Applicant: United Arab Emirates University, Al Ain (AE)

(72) Inventors: Omar M. Ali El-Agnaf, Morecambe (GB); Nishant Narayanan Vaikath, Karivellur (IN); Nour Khaled Majbour, Lattakia (SY)

(21) Appl. No.: 15/815,354

(22) Filed: Nov. 16, 2017

Related U.S. Application Data
(60) Provisional application No. 62/423,488, filed on Nov. 17, 2016.

(51) Int. Cl. C07K 16/18; G01N 33/68

(52) U.S. Cl. CPC C07K 16/18; G01N 33/6896; C07K 2317/33; G01N 2333/4703; C07K 2317/92; G01N 2800/28; C07K 2317/34

(57) ABSTRACT

The invention relates to an antibody or fragment thereof and to a method of treatment and/or diagnosis using the antibody or fragment thereof. The antibody or fragment thereof is capable of binding to human alpha-synuclein, including monomeric, oligomeric and fibril forms of alpha-synuclein; is cross reactive with beta-synuclein and gamma synuclein; and binds an epitope of human alpha synuclein comprising the sequence MPSEEGYQDYEPEA (SEQ ID NO:2). A method for diagnosing a neurodegenerative disease associated with alpha-synuclein or of detecting alpha-synuclein comprises adding the antibody or fragment thereof to a biological sample from a subject, and detecting the presence or absence of a complex formed between alpha-synuclein and the antibody or fragment.

### CROSS REFERENCE TO RELATED APPLICATION

This application claims the benefit of U.S. Provisional Application No. 62/423,488, filed Nov. 17, 2016, which is incorporated herein by reference in its entirety.

### SEQUENCE LISTING

This application contains a Sequence Listing which has been submitted electronically in ASCII format and is hereby incorporated by reference in its entirety. Said ASCII copy, created on Nov. 15, 2017, is named 9194-142164_ST25.txt and is 1,693 bytes in size.

### FIELD OF THE INVENTION

This invention is directed to alpha-synuclein antibodies or fragments thereof. The invention is also directed to the use of these antibodies and fragments in assays and in the diagnosis and treatment of alpha-synuclein disease and disorders.

### BACKGROUND

Synucleins are a family of small proteins, about 14 kDa that are expressed at high levels in nervous tissues. The three members of the family are alpha-synuclein, beta-synuclein, and gamma-synuclein.

Alpha-synuclein is expressed mainly in brain tissues and is primarily located at the presynpatic terminal of neurons. The primary structure of the human form of alpha-synuclein consists of a 140 amino acid polypeptide. The wild type sequence of human alpha-synuclein can be found in FIG. 1 (SEQ ID NO:1). Alpha-synuclein normally exists as a soluble monomeric protein but can adopt several different folded confirmations depending on its environment. Monomeric alpha-synuclein can also aggregate into oligomers and into higher molecular weight insoluble fibrils.

Diseases associated with abnormalities in synucleins are often referred to as the synucleinopathies. Synucleinopathies include neurological and neurodegenerative disease and disorders such as Parkinson's disease (PD), dementia with Lewy bodies (DLB) and multiple system atrophy (MSA). In synucleinopathies it has been shown that soluble alpha-synuclein oligomers in brain homogenates of PD and DLB are elevated compared to normal brains. In addition the neuropathologic lesions (Lewy bodies) that often characterise the end stage of PD and DLB have largely been found to be composed of fibrillar alpha-synuclein deposits.

### SUMMARY OF THE INVENTION

This invention is directed to antibodies and fragments thereof of specifically binding to human alpha-synuclein. The antibodies bind monomeric, oligomeric and fibril forms of human alpha-synuclein, are cross reactive with beta-synuclein or gamma synuclein and bind to an epitope of human alpha synuclein comprising the sequence MPSEEGYQDYEPEA (SEQ ID NO:2).

The invention is also directed to compositions comprising such an antibody or fragment thereof and to methods of using the antibody or fragment thereof to detect the presence of alpha-synuclein and their use to measure the amounts of alpha-synuclein present in a sample and in the diagnosis and/or prognosis of diseases associated with alpha-synuclein.

In further embodiments the invention is directed to compositions comprising such an antibody or fragment thereof and methods using the antibody or fragment to treat or prevent diseases associated with alpha-synuclein.

The present invention provides new tools to follow the progression of neurodegenerative diseases associated with alpha-synuclein.

### BRIEF DESCRIPTION OF THE DRAWINGS

The invention will now be described by way of example with reference to the accompanying drawings:

FIG. 1 shows the wild-type sequence of human alpha-synuclein.

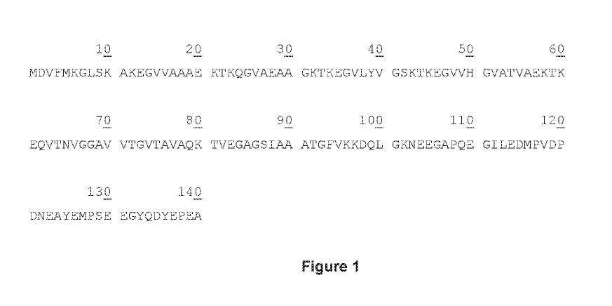

### DETAILED DESCRIPTION

The invention relates to antibodies specifically binding to human alpha-synuclein, wherein the antibody or fragment thereof:
binds monomeric, oligomeric and fibril forms of alpha-synuclein;
is cross reactive with beta-synuclein and gamma synuclein; and
   binds an epitope of human alpha synuclein comprising the sequence MPSEEGYQDYEPEA (SEQ ID NO:2).

The present invention is directed to antibodies, antigen-binding fragments which are capable of specifically recognizing alpha-synuclein. By “specifically recognizing alpha-synuclein”, “antibody specific to/for alpha-synuclein” and “antibodies specifically binding to alpha-synuclein” is meant antibodies to the native form of alpha-synuclein including those antibodies that can recognise native monomeric forms, oligomeric forms including protofibrils, and fibril forms. The antibodies may also recognise posttranslationally modified forms of alpha-synuclein.

The antibodies of the invention recognise both monomeric and aggregated forms of alpha-synuclein. Unless otherwise stated the term alpha-synuclein aggregates is intended to cover early soluble aggregates forms of alpha-synuclein (such as low and high molecular weight soluble oligomers, including protofibrils) and mature insoluble aggregates forms of alpha-synuclein (such as mature fibrils).

Fibrils are insoluble higher molecular weight aggregated forms of alpha-synuclein.

Soluble oligomeric forms of alpha-synuclein come in a variety of sizes and morphologies and includes dimer, trimers, tetramers and multimers. Protofibrils are an intermediate step in the pathway to the formation of the alpha-synuclein fibrils from the monomeric forms. When the term oligomeric forms of alpha-synuclein is used this is also intended to include the higher molecular-weight protofibrils.

The antibodies and fragments thereof have high binding affinity to human alpha-synuclein. Having a high affinity for alpha-synuclein means that the antibodies or fragments exhibit a dissociation constant, Kd of less of than 10⁻⁷M for alpha-synuclein. Preferably the antibody exhibits a Kd of less than 10⁻⁸M, or less than 10⁻⁹M, or even more preferably a Kd of less than 10⁻¹⁰M, or even less than 10⁻¹¹M.

In addition to human alpha-synuclein the antibodies also cross react with beta and gamma-synuclein. By “cross react” it is meant that the antibodies are recognise or bind beta and gamma-synuclein. The antibodies and fragments thereof can also have high binding affinity to beta and gamma-synuclein. Having a high affinity means that the antibodies or fragments exhibit a dissociation constant, Kd of less of than 10⁻⁷M for the beta and gamma-synuclein. Preferably the antibody exhibits a Kd of less than 10⁻⁸M, or less than 10⁻⁹M, or even more preferably a Kd of less than 10⁻¹⁰M, or even less than 10⁻¹¹M.

The antibodies recognise the epitope that comprises or consists of the sequence, MPSEEGYQDYEPEA (SEQ ID NO:2), corresponding to amino acids 127-140 of human alpha-synuclein. In one embodiment the antibodies bind more strongly to the linear epitope than to other linear peptide epitopes of alpha-synuclein of the same length.

The antibodies of the invention may also bind to phosphorylated forms of alpha-synuclein. In one embodiment the antibodies can bind to the alpha-synuclein phosphorylated at Ser129. The antibodies may bind to nitrated forms of alpha-synuclein. The antibodies may also bind to oxidised forms of alpha-synuclein.

A fragment thereof of the antibodies means alpha-synuclein binding fragments thereof, i.e. fragments having the same binding characteristics as the antibody according to the invention, namely binds to an epitope of alpha-synuclein comprising the sequence MPSEEGYQDYEPEA (SEQ ID NO:2) and has specificity for alpha-synuclein monomers, oligomers and fibrils, and does substantially cross react with beta-synuclein and gamma-synuclein. For convenience when the term antibody is used, fragments thereof exhibiting the same characteristic are also being considered.

The antibodies of the invention also do not cross react with other amyloid proteins. By “do not cross react” or “not cross reactive” it is meant that the antibodies are not significantly cross reactive or do not significantly recognise the other amyloid proteins.

The antibodies of the invention exhibit low binding affinity to these other amyloid proteins. By other amyloid proteins it includes, but is not limited to, IAPP (islet amyloid polypeptide), β-amyloid monomers, Tau, ABri, and synthetic peptides such as Aβ-42. The binding affinity of the antibodies can be at least 100 times less to one or more of these other amyloid peptide/proteins than that to human alpha-synuclein.

The binding affinities of the antibodies can be determined by using a variety of methods recognised in the art including, isothermal calorimetry and surface plasmon resonance based approaches. Binding can also be evaluated using immunoassays such as ELISA or RIAs. Preferably the binding affinity is determined using surface plasmon resonance assays using a BIACore™ X-100.

Examples of antibodies according to the invention have been developed by traditional hybridoma techniques. In one embodiment the antibodies are monoclonal. Preferably the antibodies are mouse monoclonal antibodies to alpha-synuclein.

The antibody can be any class or isotype antibody, for example IgM or IgG. Preferably the antibody is IgG antibody.

In another aspect of the invention the antibodies can be used as diagnostic tools for detecting the presence of alpha-synuclein in a sample. The antibodies may be used for monitoring and/or diagnosing a synucleinopathy, such as a neurological or neurodegenerative disorder associated with alpha-synuclein in a subject.

These antibodies may be suitable as diagnostic tools for synucleinopathies such as neurological or neurodegenerative disorders associated with alpha-synuclein, including but not limited to Parkinson's disease, dementia with Lewy bodies and other alpha-synuclein related neurodegenerative disorders.

In one embodiment the invention relates to a method of detecting alpha-synuclein comprising the steps of:
adding the antibody or fragment thereof to a biological sample; and
detecting the presence of a complex formed between alpha-synuclein and the antibody or fragment.

The detection of complexes indicates the presence of alpha-synuclein in the sample.

The method can further comprise the step of measuring the level of complex formed and comparing the levels to a reference level. The reference level will typically be calculated from a sample from an individual known not to have an alpha-synuclein pathology (a “normal individual”) or from an earlier test of a sample taken from the same individual being tested.

The method can detect monomer, oligomers and fibrils forms of alpha-synuclein. The method can be used to determine the total amount of alpha-synuclein in a sample.

The method can be carried out in vitro on a tissue or biological fluid sample. The sample obtained from the individual to be tested, can for example be cerebrospinal fluid, (CSF), blood, urine, saliva, or brain, gut, colon, skin or salivary gland tissues. In particularly preferred methods the sample is a CSF sample. In another preferred method the sample is a brain tissue sample.

The sample is combined with the antibody for a time and under conditions effective to allow binding of the antibody to alpha-synuclein in the sample. The sample may be processed prior to being assayed using standards methods. In one embodiment the tissue sample under goes no pre-treatment before testing with the antibody. By pre-treatment it is meant the tissue sample obtained is not subjected to any treatment such as, autoclaving, formic acid and/or proteinase K treatment.

Standard methods known in the art may be used to detect and/or measure the level of the complex formed between the antibodies and alpha-synuclein in the sample.

Analysis for the presence of alpha-synuclein can be conducted by a method such as radioimmunoassay, an enzyme-linked immunosorbant assay (ELISA), a sandwich immunoassay, a fluorescent immunoassay, a precipitation reaction, a gel immunodiffusion assay, an agglutination assay, a protein A immunoassay, an immunoelectrophoresis assay, an electrophoresis, western blotting. Other suitable techniques able to measure and/or detect the presence of alpha-synuclein in the sample to be tested can also be used.

In one embodiment the antibodies may be coated onto a surface, such as a microwell plate or diagnostic test strip, and the sample added to the antibody and allowed to combined under conditions effective to allow binding. The presence of the complex can then be detected.

In a preferred method an ELISA assay is used to detect and/or quantify the total amount of alpha-synuclein. In one embodiment the invention is directed to a sandwich ELISA comprising adding the sample to be tested to a microplate, where the surface of the microplate has been coated with an antibody; allowing any alpha-synuclein present in the sample to bind to the antibodies; and detecting the presence of any antibody/alpha-synuclein complexes. Detection can be carried out using labelled antibodies that bind alpha-synuclein. The antibodies of the invention may be used as the capture and/or the detection antibody in the ELISA.

The methods can be used for the diagnosis of neurodegenerative diseases and/or monitoring the progression of a neurodegenerative disease. The amount and/or size of any alpha-synuclein can be detected.

The neurodegenerative disease can include but is not limited to Parkinson's disease, dementia, Alzheimer's disease, Down's syndrome, multiple-system atrophy, psychosis, schizophrenia or Creutzfeldt-Jakob disease. The dementia may be dementia with Lewy bodies.

In one embodiment the method of diagnosing a neurodegenerative disease associated with alpha-synuclein comprises: adding an antibody of the invention to a sample from an individual; detecting the presence of a complex formed between the alpha-synuclein and the antibody; and determining whether or not the individual has a neurodegenerative disease associated with alpha-synuclein.

Determining whether or not the individual has a neurodegenerative disease can comprise comparing the levels of the complex formed in a sample with a reference level and determining whether the levels of complexes formed in the sample have decreased or increased relative to a reference level.

A change in the level of alpha-synuclein as compared to the reference level indicates that the individual has a neurodegenerative disease.

The method can further comprise administering to the individual a therapeutically effective amount of an agent to treat the neurodegenerative disease.

In a further embodiment a method of monitoring the progress of a neurodegenerative disease associated with alpha-synuclein comprises: adding an antibody of the invention to a sample from an individual; detecting the presence of a complex formed between the alpha-synuclein and the antibody; and comparing the levels of the complex formed in a sample with a reference level.

The method can further comprise altering the treatment regime of the individual based on the comparison of the detected levels of complex with the reference level. The treatment regime can be altered by changing the drugs administered to treat the disease and/or changing the frequency and/or dose of the drug administered, depending on the progress of the disease. An increased level of the complex compared to a base line level will typically indicate that the individual has or is in the process of developing an alpha-synuclein pathology. The base line level will typically be calculated from a sample from an individual known not to have an alpha-synuclein pathology (a “normal individual”) or from an earlier test of a sample taken from the same individual being tested.

A correlation has been shown to exist between CSF alpha-synuclein levels and disease severity. Detecting the presence and/or amount of the synuclein in the sample can be used to follow the progression and or severity of a neurodegenerative disease, in particular for using the antibodies as a biomarker in Parkinson diseases and other diseases associated with alpha-synuclein pathologies.

In one embodiment of the invention the antibodies are used to diagnose whether an individual has Parkinson's disease. A CSF sample is taken from the patient. Antibodies are contacted with the sample in conditions effective to allow complexes to form between the antibodies and alpha-synuclein present in the sample. The presence of the antibody complexes can then be detected. The amount of complexes formed can be measured and compared to a reference level.

In one embodiment of the invention the antibodies can be used in an ELISA to measure alpha-synuclein in CFS. The antibodies can be used to measure alpha-synuclein in a sample with high sensitivity and specificity compared to ELISA using other antibodies. In particular an ELISA using the antibodies of the invention has a higher sensitivity and specificity to detect alpha-synuclein in CSF as compared to currently known ELISA. The antibodies of the invention can be used as a capture antibody or a detection antibody in an ELISA.

The methods can be used to monitor the effectiveness of a therapeutic agent, by using the results of the analysis undertaken. An effective therapeutic agent can be determined as one that causes a change in the amount of alpha-synuclein present in a sample taken, as compared to a reference value. The reference value may reflect the amount of alpha-synuclein in the patient before treatment, or may represent a typical amount of alpha-synuclein to be found in untreated patients.

The antibodies may be labelled with a detectable label. The label will be one that allows detection of the antibody when bound to the alpha-synuclein aggregates. Detectable labels include, but are not limited to fluorescent labels, radioactive labels, enzymes and contrast agents. Suitable radiolabels include those such as F¹⁸, I¹²³, In¹¹¹, I¹³¹, C¹⁴, H³, Tc^{99m}, P³², I¹²⁵ and Gallium 68. Suitable fluorescent labels can include fluorescein and rhodamine. Suitable contrast agents include: rare earth ions such as gadolinium (Gd), dysprosium and iron, and magnetic agents. Other labels include nuclear magnetic resonance active labels, positron emitting isotopes detectable by a PET scanner, chemiluminescent and enzymatic markers.

The antibodies can be labelled by standard techniques.

In another aspect of the invention the antibodies can be used as an imaging agent. In particularly the antibodies can be used for detecting and localization and/or quantitation of alpha-synuclein in human tissues.

The invention provides a method of imaging alpha-synuclein aggregates, comprising detecting the binding of the antibody to alpha-synuclein aggregates.

In one embodiment antibodies of the invention can be contacted with a sample and then the antibody in the sample that has bound to alpha-synuclein can be detected. The antibody is preferably a labelled antibody. The presence or absence of alpha-synuclein may be detected in the brain in vivo using any suitable imaging techniques. In such in vivo methods, the method may further comprise administering the antibody to an individual and detecting the antibody.

Suitable imaging techniques include positron emission tomography (PET), gamma-scintigraphy, magnetic resonance imaging (MRI), functional magnetic resonance imaging (FMRI), magnetoencephalography (MEG), and single photon emission computerized tomography (SPECT).

The presence or absence of alpha-synuclein may also be detected in vitro, for example in tissue samples, such as a brain section. In such embodiments suitable imaging techniques may also include electron microscopy, confocal microscopy or light microscopy.

The number and/or size of alpha-synuclein aggregates present in the brain of an individual correlates with the progression of the alpha-synuclein associated disease. An increase in the size or number of alpha-synuclein aggregates indicates a progression of the disease, whilst a decrease in the size or number of alpha-synuclein aggregates indicates a regression of the disease. The antibodies of the invention bind fibril forms of alpha-synuclein and therefore can be used to detect the presence of aggregates of alpha-synuclein subjects.

The invention also relates to a kit comprising an antibody according to the invention for carrying out the diagnostic methods. The antibody may be an intact immunoglobulin molecule or fragment thereof such as Fab, F(ab)2 or Fv fragment. The antibody may be labelled as described above. The kit can be for use in a method of determining whether an individual has a neurodegenerative disease.

The kit may additionally comprise one or more other reagents or instruments which enable any of the methods to be carried out. Such reagents or instruments including, but not limited to one or more of the following, suitable buffers, means to obtain a sample from an individual, a support comprising wells on which quantitative reactions can be done. The kit may optionally comprise instructions for carrying out the methods above.

In one embodiment of the invention the antibody and fragment thereof can be used as a medicament.

The invention relates to antibodies or fragments thereof for use in the treatment of a synucleopathies, such as a neurodegenerative disorder associated with alpha-synuclein in an subject.

The invention also relates to a method of treating a neurodegenerative disorder with alpha-synuclein pathology in an subject, comprising administering to the subject a therapeutically effective amount of the antibody or fragment thereof.

The neurodegenerative disorder can include but is not limited to Parkinson's disease, dementia, Alzheimer's disease, Down's syndrome, multiple-system atrophy, psychosis, schizophrenia or Creutzfeldt-Jakob disease. The dementia may be dementia with Lewy bodies.

Alpha-synuclein aggregation may be reduced or inhibited by the administration of an antibody or fragment thereof. The antibody may be administered to a sample comprising soluble synuclein species or directly to a subject. The subject may be a human.

The antibody may be administered directly to the site of alpha-synuclein aggregate deposit, e.g. a Lewy body, typically by injection into a blood vessel supplying the brain or into the brain itself.

The terms ‘treatment’ and “treating” and the like, is intended to include curing, relieving, reversing, alleviating, managing or delaying the onset, of the condition, or to reduce the risk of developing or worsening the condition. The terms are also intended to include palliative, prophylactic and preventative treatment of the condition.

In one embodiment of the invention a pharmaceutical composition comprises the antibody or fragment thereof and a pharmaceutically acceptable diluent or carrier.

In general, the nature of the carrier will depend on the particular mode of administration being employed. Pharmaceutical forms include solid, solutions and suspensions. Suitable pharmaceutical carriers include inert diluents or fillers, water and various organic solvents. Compositions may also include additional ingredients such as flavourings, binders & excipients.

Forms suitable for oral administration include tablets, capsules, pills, powders, sustained release formulations, solutions, and suspensions. Forms suitable for parental injection include sterile solutions, suspensions or emulsions.

Exemplary parenteral administration forms include suspensions or solutions in sterile aqueous solutions, for example aqueous propylene glycol or dextrose solutions. Such dosage forms can be suitably buffered, if desired.

Exemplary oral forms such as tablets may include: disintegrants such as starch, alginic acid and complex silicates; binding agents such as sucrose, gelatin and acacia; and lubricating agents such as magnesium stearate, sodium lauryl sulfate and talc. Solid compositions may also include soft and hard gelatin capsules. Preferred materials include lactose, milk sugar and high molecular weight polyethylene glycols.

Methods of preparing various pharmaceutical compositions are well known to those skilled in the art.

The invention also relates to the antibody and fragment in combination with one or more further therapeutic agents

### Examples

### Preparation of Antibodies

Full length recombinant human alpha-synuclein fibrils were used as the antigen to generate the antibodies. The antigens were repeatedly used to immunize female BALB/C mice subcutaneously. Mice with appropriate plasma titers were euthanized and splenocytes were extracted and fused with Sp2/0 myeloma cells and then seeded in HAT (hypoxantin, aminopterine, and thymidine) selective medium to generate antibody-producing hybridomas according to standard techniques. After 10 days, the medium was replaced with hypoxantin, thymidine medium and 5 days later the culture supernatants were tested for secreted anti-α-syn antibodies using indirect ELISA. Monoclonal antibodies were produced, purified and thoroughly characterized.

A summary of the characteristics is provided below:

TABLE 1

| | |
|---|---|
| Description | Mouse monoclonal antibody to alpha- |
| | synuclein |
| Species reactivity | Human |
| Isotype | IgG |
| Molecular weight | ~150 kDa |
| Hybridoma type | Mouse myeloma hybridoma |
| Epitope recognised | Amino acid residues 127-140 of human |
| | alpha-synuclein (MPSEEGYQDYEPEA) |
| | (SEQ ID NO: 2) |
| Conformation of epitope | Linear |
| Modifications recognised | Alpha-synuclein monomers, oligomers and |
| | fibrils |
| | Nitrated, oxidised and phosphorylated |
| | alpha-synuclein |
| Cross Reactivity | Cross-reacts with beta and gamma |
| | synuclein |

1. An antibody or fragment thereof capable of binding to human alpha-synuclein, wherein the antibody or fragment thereof:
binds monomeric, oligomeric and fibril forms of alpha-synuclein;
is cross reactive with beta-synuclein and gamma synuclein; and
binds an epitope of human alpha synuclein comprising the sequence MPSEEGYQDYEPEA (SEQ ID NO:2).

2. An antibody or fragment thereof according to claim 1, wherein the antibody or fragment thereof has a dissociation constant, Kd, of less than 10⁻⁷ M for alpha-synuclein.

3. An antibody or fragment thereof according to claim 1, wherein the antibody does not cross react with IAPP (islet amyloid polypeptide), β-amyloid monomers, Tau or ABri.

4. An antibody or fragment thereof according to claim 1, wherein the antibody is a monoclonal antibody.

5. An antibody or fragment thereof according to claim 1, wherein the antibody is an IgG antibody.

6. An antibody or fragment thereof according to claim 1, wherein the antibody has a molecular weight of about 150 kDa.

7. An antibody or fragment thereof according to claim 1, wherein the antibody comprises a detectable label.

8. An antibody or fragment thereof according to claim 7, wherein the detectable label is a fluorescent label, radioactive label, an enzyme or a contrast agent.

9. A method for diagnosing a neurodegenerative disease associated with alpha-synuclein or of detecting alpha-synuclein, the method comprising:
adding the antibody or fragment thereof according to claim 1 to a biological sample from a subject, and
detecting the presence or absence of a complex formed between alpha-synuclein and the antibody or fragment.

10. A method for preventing or treating a neurodegenerative disorder with alpha-synuclein pathology in a subject comprising administrating an antibody or fragment thereof according to claim 1 to the subject.

11. A method according to claim 10, wherein the neurodegenerative disorder is Parkinson's disease, dementia with Lewy Bodies, Alzheimer's disease, multiple system atrophy, psychosis, schizophrenia or Creutzfeldt-Jakob disease.
